(19) United States
(12) Patent Application Publication
(10) Pub. No.: US 2026/0026936 A1
(43) Pub. Date: Jan. 29, 2026

(54) Method and Apparatus for Collapsing a Prosthetic Heart Valve

(71) Applicant: Cephea Valve Technologies, Inc., Abbott Park, IL (US)

(72) Inventors: Randolf Von Oepen, Aptos, CA (US); Russell Shelton, San Jose, CA (US); Gregory M. Hyde, Menlo Park, CA (US)

(73) Assignee: Cephea Valve Technologies, Inc., Abbott Park, IL (US)

(21) Appl. No.: 19/345,724

(22) Filed: Sep. 30, 2025

Related U.S. Application Data
(63) Continuation of application No. 17/719,645, filed on Apr. 13, 2022.
(60) Provisional application No. 63/174,699, filed on Apr. 14, 2021.

(51) Int. Cl. A61F 2/24

(52) U.S. Cl. CPC A61F 2/2439; A61F 2/2418; A61F 2250/001; A61F 2250/0018

(57) ABSTRACT

A prosthetic heart valve system includes a collapsible and expandable outer frame extending from an atrial end to a ventricular end, the atrial and ventricular ends coupled by a central portion, the outer frame including a plurality of suture receiving rings formed around a circumference of the ventricular end. The system further includes a collapsible and expandable inner frame positioned radially inward of and coupled to the outer frame. The system further includes a prosthetic valve assembly coupled to, and positioned radially inward of, the inner frame, and a strand extending from a first free end to a second free end. A middle portion of the strand passes through each of the suture receiving rings in a delivery configuration of the prosthetic heart valve system. The first and second free ends are configured to be pulled simultaneously by a user to radially collapse the ventricular end of the outer frame.

### CROSS-REFERENCE TO RELATED APPLICATIONS

This application is a continuation application of U.S. application Ser. No. 17/719,645, filed Apr. 13, 2022, and claims the benefit of the filing of U.S. Provisional Application No. 63/174,699 filed Apr. 14, 2021, the disclosures of each of which are hereby incorporated by reference herein in their entireties.

### BACKGROUND OF THE DISCLOSURE

Valvular heart disease, and specifically aortic and mitral valve disease, is a significant health issue in the United States. The mitral valve lies between the left atrium and the left ventricle of the heart. Various diseases can affect the function of the mitral valve, including degenerative mitral valve disease and mitral valve prolapse. These diseases can cause mitral stenosis, in which the valve fails to open fully and thereby obstructs blood flow, and/or mitral insufficiency, in which the mitral valve is incompetent and blood flows passively in the wrong direction.

Many patients with heart disease, such as problems with the mitral valve, are intolerant of the trauma associated with open-heart surgery. Age or advanced illness may have impaired the patient's ability to recover from the injury of an open-heart procedure. Additionally, the high costs associated with open-heart surgery and extra-corporeal perfusion can make such procedures prohibitive.

Patients in need of cardiac valve repair or cardiac valve replacement can be served by minimally invasive techniques. In many minimally invasive procedures, small devices are manipulated within the patient's body under visualization from a live imaging source like ultrasound, fluoroscopy, or endoscopy. Minimally invasive cardiac procedures are inherently less traumatic than open procedures and may be performed without extra-corporeal perfusion, which carries a significant risk of procedural complications.

During minimally invasive procedures for mitral valve replacement, the mitral valve prosthesis generally must be collapsed into a small delivery device for placement within the native mitral valve orifice. Typically, prosthetic heart valves that are collapsible are capable of expanding or re-expanding by either balloon expansion or self-expansion. Conventional methods of placement of a prosthetic valve in the native valve orifice include navigating the prosthetic valve to the native valve with a delivery device and expanding the valve when in the desired location. A prosthetic valve may include an outer frame or anchor assembly having anchors and/or retention members extending radially outward from the prosthetic heart valve frame for securing the prosthetic valve in place. The prosthetic valve may include anchoring mechanisms such as radial force, pinching the native annulus between two disks, hooks for penetrating the surrounding native tissue, and other mechanisms or combinations thereof. In some configurations, once the prosthetic valve has been partially or fully deployed, the prosthetic heart valve may not be movable or re-positionable, giving a surgeon or operator only one opportunity to accurately place and deploy the prosthetic valve. A means for collapsing a prosthetic heart valve after it has been at least partially expanded is therefore desired.

### BRIEF SUMMARY OF THE DISCLOSURE

Disclosed herein is an apparatus and method for at least partially collapsing a prosthetic heart valve after the prosthetic valve has been at least partially deployed from a delivery device. The prosthetic heart valve includes a compression resistant member disposed within the lumen of the prosthetic valve, the compression resistant member defining a lumen therethrough and configured to resist buckling due to an axial force. The prosthetic heart valve further includes a plurality of suture receiving rings formed around the circumference of the ventricular end of the prosthetic heart valve. The prosthetic valve may be loaded into the delivery device with a strand extending through the lumen of the compression resistant member and around the circumference of the ventricular end, passing through the plurality of suture receiving rings. The first and second ends of the strand may extend toward a user or operator through the atrial end of the prosthetic valve. The first and second ends may be accessible to a user or operator and configured to be tensioned to apply a radially inward force to the suture receiving rings on the ventricular end of the prosthetic valve. The prosthetic valve may be delivered to the native valve, at least partially deployed, and re-collapsed by tensioning the strand to adjust the prosthetic valve's position in the native valve. After placement of the prosthetic valve is finalized, the strand may be removed by pulling on one end of the strand.

According to a first aspect of the disclosure, a prosthetic heart valve system may include a collapsible and expandable outer frame, a collapsible and expandable inner frame, a prosthetic valve assembly, and a strand. The outer frame may extend from an atrial end to a ventricular end. The atrial and ventricular ends may be coupled by a central portion. The outer frame may include a plurality of suture receiving rings formed around a circumference of the ventricular end. The inner frame may be positioned radially inward of the outer frame and may be coupled to the outer frame. The valve assembly may be coupled to and may be positioned radially inward of the inner frame. The strand may extend from a first free end to a second free end. A middle portion of the strand may pass through each of the suture receiving rings in a delivery configuration of the prosthetic heart valve system. The first and second free ends may be configured to be pulled simultaneously by a user to radially collapse the ventricular end of the outer frame.

According to another embodiment of the disclosure, a prosthetic heart valve system may include a collapsible and expandable outer frame, a collapsible and expandable inner frame, a prosthetic valve assembly, a first strand, and a second strand. The outer frame may extend from an atrial end to a ventricular end. The atrial and ventricular ends may be coupled by a central portion. The outer frame may include a plurality of suture receiving rings formed around a circumference of the ventricular end. The inner frame may be positioned radially inward of the outer frame and may be coupled to the outer frame. The prosthetic valve assembly may be coupled to and may be positioned radially inward of the inner frame. The first strand may extend from a first free end to a second free end. A middle portion of the first strand may pass through a first group of the suture receiving rings in a delivery configuration of the prosthetic heart valve system. The second strand may extend from a first free end to a second free end. A middle portion of the second strand may pass through a second group of the suture receiving rings in the delivery configuration of the prosthetic heart valve system. The first and second ends of the first and second strands may be configured to be simultaneously pulled by a user to radially collapse the ventricular end of the outer frame.

### BRIEF DESCRIPTION OF THE DRAWINGS

FIG. 1A is a perspective view of a prosthetic heart valve frame according to an embodiment of the disclosure.

FIG. 1B is a side view of an outer frame of the prosthetic heart valve frame of FIG. 1A.

FIG. 1C is a side view of an inner frame of the prosthetic heart valve frame of FIG. 1A.

FIG. 1D is a perspective view from the ventricular side of a prosthetic heart valve according to an embodiment of the disclosure.

FIGS. 2A-D are schematic views of the prosthetic heart valve of FIGS. 1A-C being deployed from a delivery device in a native mitral valve orifice according to an embodiment of the disclosure.

FIG. 3 is a highly schematic cross section of a prosthetic heart valve according to an embodiment of the disclosure.

FIGS. 4A-B are schematic side views of the prosthetic heart valve of FIG. 3.

FIGS. 5A-B are highly schematic cross sections of a prosthetic heart valve according to other embodiments of the disclosure.

### DETAILED DESCRIPTION

As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus. Collapsible/expandable prosthetic heart valves may be used to replace any heart valve including a mitral valve, tricuspid valve, aortic valve, or pulmonary valve. However, the devices disclosed herein may be particularly suited for replacement of the mitral and tricuspid valves.

This disclosure includes an apparatus and method for collapsing a prosthetic mitral valve after the prosthetic mitral valve has been at least partially deployed from a delivery device. It should be noted that the apparatus described herein is not limited to use with a prosthetic mitral valve, but may be used with any prosthetic heart valve or collapsible apparatus. For example, the device described herein may be used on the same or similar structures that may be radially collapsed, such as a bag, case, covering, or any other collapsible structure. As used herein, the terms “substantially,” “generally,” “approximately,” and “about” are intended to mean that slight deviations from absolute are included within the scope of the term so modified. As used herein, the valve may assume an “expanded condition” and a “collapsed condition,” which refer to the relative radial size of the valve. It should be noted that in different embodiments described throughout the disclosure, like numbers refer to like elements unless otherwise indicated.

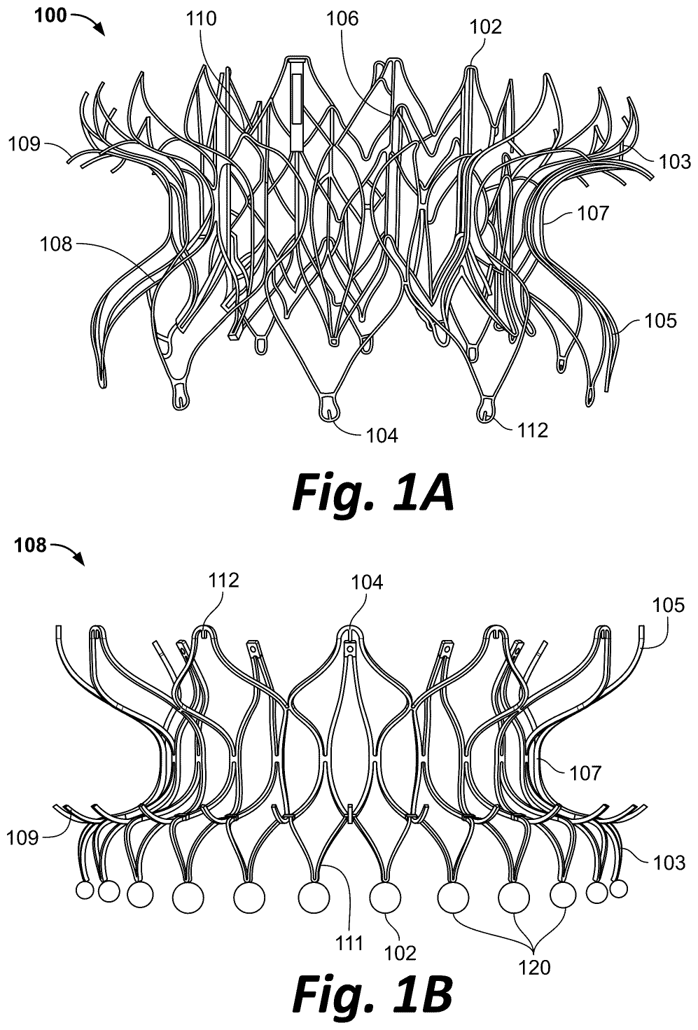

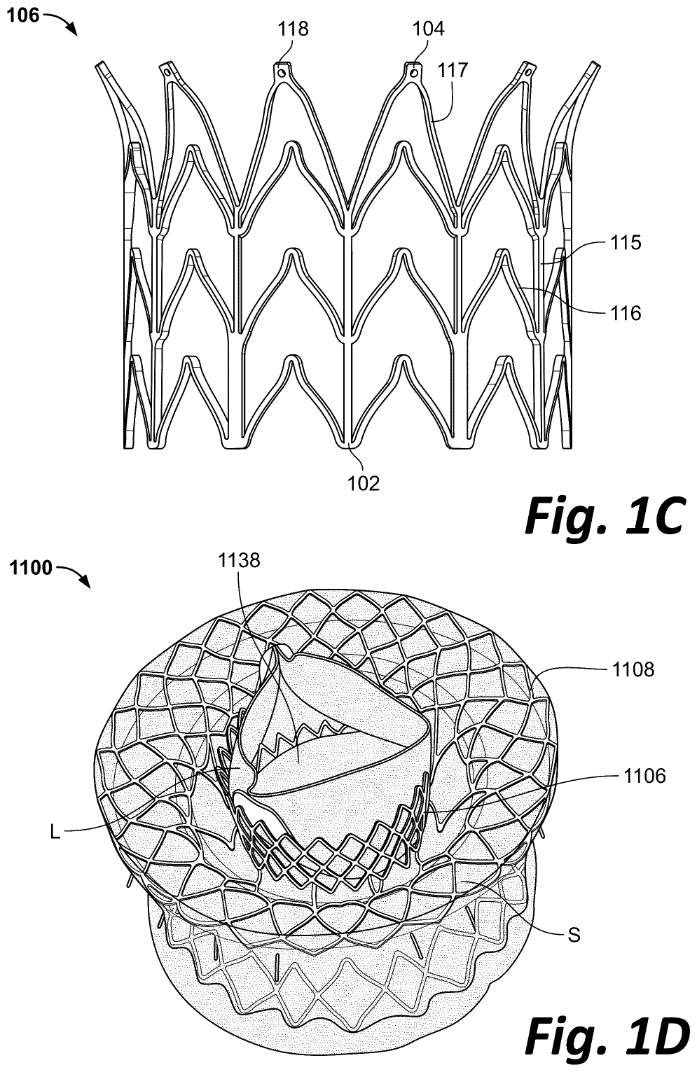

FIG. 1A illustrates a prosthetic heart valve 100 in accordance with some embodiments of the disclosure. It should be understood that the prosthetic heart valve 100 illustrated in FIG. 1A omits certain features that would typically be included, such as a valve assembly to assist in controlling blood flow through the prosthetic heart valve, and interior and/or exterior fabrics or tissue skirts to assist with providing a seal around the prosthetic heart valve and/or with enhancing tissue ingrowth to fix the prosthetic heart valve within the native heart valve over time. However, for purposes of simplicity, the prosthetic leaflet(s) and skirt(s) are omitted from FIG. 1A for clarity of illustration.

The prosthetic heart valve 100 extends from a ventricular end 102 to an atrial end 104. The valve 100 includes an inner frame 106 and an outer frame or anchor assembly 108 positioned radially outward of the inner frame 106. The outer frame 108 may be primarily for anchoring the prosthetic heart valve 100 within the native heart valve annulus, while the inner frame 106 may be primarily for holding the prosthetic valve assembly in the desired position and orientation. The inner and outer frames 106, 108 can be assembled to form prosthetic valve frame 110, a support structure configured to fit within a native valve annulus.

The outer frame 108 is illustrated more clearly in FIG. 1B, isolated from other components of the prosthetic heart valve 100. Outer frame 108 is illustrated in a vertically flipped orientation in FIG. 1B compared to FIG. 1A. The outer frame 108 includes a ventricular portion or anchor 103 near the ventricular end 102, an atrial portion or anchor 105 near the atrial end 104, and a central portion 107 coupling the ventricular and atrial portions 103, 105. The ventricular portion 103 may be configured and adapted to be disposed on the ventricular side of a native valve annulus, and may flare radially outwardly from the central portion 107. The atrial portion 105 may be configured and adapted to be disposed on an atrial side of the native valve annulus, and may also flare radially outwardly from the central portion 107. The central portion 107 may be configured to be situated in the valve orifice, for example in contact with the native valve annulus, and may have the shape of a narrow waist between the ventricular and atrial portions 103, 105. In use, the ventricular portion 103 and atrial portion 105 effectively clamp the native valve annulus on the ventricular and atrial sides thereof, respectively, holding the prosthetic heart valve 100 in place. The outer frame 108 may include ventricular v-shaped elements 111 where the apex of each v-shaped element 111 is located at the ventricular end 102. A suture receiving structure, e.g., suture receiving ring 120, may be coupled to the apex of each v-shaped element 111. Each suture receiving ring 120 may be configured to receive a suture or strand, as described below in more detail. It is contemplated that the suture receiving rings 120 may be any suitable shape for receiving a suture, such as circular, an oval, an open-ended C-shape, or the like, and although it is preferable that the structure will fully circumscribe a suture received therein, in some embodiments the structure need not fully circumscribe the suture received therein. In some examples, the suture receiving rings 120 may be formed monolithically with the outer frame 108 of the prosthetic valve 100. In other examples, the suture receiving rings 120 may be coupled to the outer frame 108 of the prosthetic valve 100 by any suitable means, such as welding or the like.

The outer frame 108 may further include barbs or tines 109 flaring radially outward from the outer frame 108 at the ventricular portion 103 to anchor prosthetic heart valve 100 in the native heart valve. Tines 109 may be spaced approximately equal distances apart around the outer circumference of the outer frame 108. When the outer frame 108 is in the expanded condition, the tines 109 may hook upwardly toward the atrial end 104 and terminate in a free end adapted to engage with and/or pierce native tissue. The free ends of the tines 109 may be blunt or sharp. The outer frame 108 may further include tabs 112 at the atrial end 104 of the outer frame 108. The tabs 112 may be evenly spaced around the circumference of the atrial end 104 of the outer frame 108. It should be noted that the tabs 112 in FIG. 1A are shown surrounded by a closed stent structure, while the tabs 112 in FIG. 1B are shown without any similar closed stent structures. In use, suture loops of a delivery device may hook onto or over the tabs 112 to keep the prosthetic heart valve 100 tethered to the delivery device via the suture loops. When it is desired to release the prosthetic heart valve 100 from its connection to the delivery device, the suture loops may be advanced distally to slide off the tabs 112, releasing the connection. The outer frame 108 may be formed of any suitable material. In some embodiments, the outer frame 108 may be formed of a shape-memory material, such as nickel titanium alloys, including nitinol. The outer frame 108 may be laser-cut from a tube of nitinol, and then shape-set so that it is biased to the expanded condition shown in FIG. 1B. However, other materials and methods of forming the outer frame 108 may be suitable.

As illustrated in FIG. 1A, the inner frame 106 may be positioned radially within the outer frame 108 when the inner and outer frames are assembled together. The inner frame 106 is illustrated in FIG. 1C isolated from other components of the prosthetic heart valve 100. One or more prosthetic leaflets may be coupled to the inner frame 106 to allow unidirectional flow of blood through the prosthetic valve assembly from the atrial end 104 toward the ventricular end 102 of the prosthetic heart valve 100. Inner frame 106 is illustrated in a vertically flipped orientation in FIG. 1C compared to FIG. 1A. Inner frame 106 may include twelve longitudinal struts 115, with three rows of twelve v-shaped members 116. However, in other embodiments, more or fewer longitudinal struts 115 may be included, and more or fewer rows of v-shaped members 116 may be included. In other embodiments, inner frame 106 may be formed of diamond-shaped cells without longitudinal struts. In addition, v-shaped coupling members 117 may extend from each adjacent pair of longitudinal struts 115. These v-shaped coupling members 117 may have half-diamond shapes with the apex of each half-diamond shape including an aperture 118, the v-shaped coupling members 117 generally flaring radially outwardly in the expanded condition of inner frame 106. The inner frame 106 may be configured to expand circumferentially (and radially) while maintaining the same (or about the same) axial dimension (e.g., be non-foreshortening) as the prosthetic heart valve 100 expands from the collapsed delivery condition to the expanded condition. The axial struts 115 may contribute to this non-foreshortening functionality. By being non-foreshortening, the inner frame 106 may prevent (or reduce) strain from being placed on the prosthetic leaflets when the inner frame 106 transitions between the collapsed and expanded conditions. Thus, while the outer frame 108 may be designed to be foreshortening, the inner frame 106 may be designed so as to be substantially non-foreshortening. However, as noted above, inner frame 106 in other embodiments may be foreshortening, for example if formed of diamond-shaped cells. The inner frame 106 may couple to the outer frame 108 via the v-shaped coupling members 117. For example, the v-shaped coupling members 117 may be positioned in contact with generally similar shaped coupling members 119 on the outer frame 108 (best shown in FIG. 1B), and a suture, rivet or other fastener may pass through apertures of the coupling members 117, 119. The inner frame 106 may be formed of any suitable material. In some embodiments, the inner frame 106 may be formed of a shape-memory material, such as nickel titanium alloys, including nitinol. The inner frame 106 may be laser-cut from a tube of nitinol, and then shape-set so that it is biased to the expanded condition shown in FIG. 1C. However, other materials and methods of forming the inner frame 106 may be suitable.

In some embodiments, the valve 100 may include a skirt covering the inner and/or outer surfaces of the inner frame 106 and/or the outer frame 108, and one or more leaflets positioned within a central channel of the frame 110 (and specifically the inner frame 106). An example of such a valve is shown in FIG. 1D, which illustrates the ventricular side of a prosthetic heart valve 1100. Briefly, prosthetic heart valve 1100 includes a plurality of prosthetic leaflets L coupled to the interior of an inner frame 1106, with the prosthetic leaflets L forming a valve assembly that is shown in FIG. 1D in an open condition. Although three prosthetic leaflets L are shown, it should be understood that in other embodiments fewer or more than three prosthetic leaflets may be provided. The inner frame 1106 may be positioned radially within, and coupled to, an outer frame 1108. Further, FIG. 1D illustrates a skirt S, which may be formed of fabric, tissue, or combinations thereof, on the inner frame 1106 and/or the outer frame 1108. The skirt S may be formed of a single piece of material or multiple pieces of material, and may extend over any one or more of the luminal and abluminal surfaces of the inner frame 1106 and the outer frame 1108. It should be understood that the inner frame 1106 may be substantially similar or identical to inner frame 106, while the outer frame 1108 may be substantially similar or identical to outer frame 108.

Referring back to FIGS. 1A-C, the frame 110 may be configured to collapse to reduce an outer diameter of the frame 110 when the frame 110 is loaded into a loading device and/or delivery system. When the frame 110 is in an expanded condition, the outer frame 108 fully extends radially outward, as shown in FIGS. 1A-B. The valve 100 is naturally in an expanded state when no force is applied to the frame 110. When the valve 100 is in a collapsed condition, the frame 110 may at least partially collapse radially inward. The valve 100 may be placed in a collapsed condition by applying pressure on the outer frame 108 in a radially inward direction. In a collapsed condition, the valve 100 may have a higher degree of potential energy (e.g., is spring loaded) compared to when in the expanded condition. Although not shown, the prosthetic valve 100 may include a spacer or balloon disposed in the center of the prosthetic valve 100 (e.g., radially inward of the inner frame 106) when the prosthetic valve 100 is collapsed to promote a uniform folding of the prosthetic valve 100. The spacer may be removed after the prosthetic valve 100 is collapsed and loaded into the delivery device.

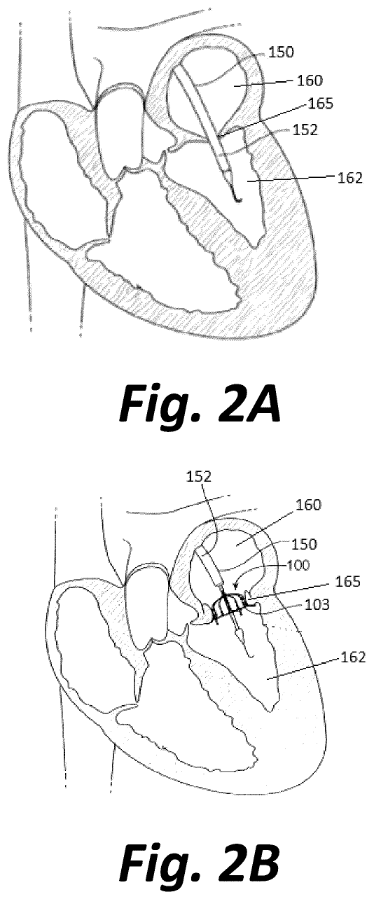

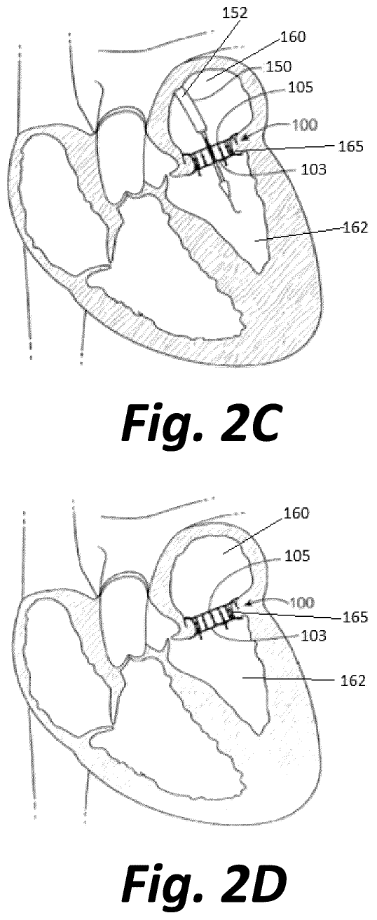

After the valve 100 is collapsed and loaded into a delivery device, the delivery device may be navigated through the patient to transport the valve 100 to the native valve orifice. For example, the valve 100 may be delivered to the native valve orifice transseptally. FIGS. 2A-D illustrate the deployment of the valve 100 from a delivery device 150. It should be noted that FIGS. 2A-D show a transseptal delivery after the delivery device 150 has been passed through the atrial septum and reached the left atrium 160, although the actual crossing of the atrial septum is not actually illustrated in FIGS. 2A-D. Thus, some parts of the delivery device are omitted for case of illustration. The term “proximal” as used herein may refer to a direction in which the delivery device 150 extends toward a user or operator of the device, and the term “distal” as used herein may refer to a direction in which the delivery device 150 extends away from a user or operator. As shown in FIG. 2A, the delivery device 150 having the prosthetic heart valve 100 disposed therein may be advanced from the left atrium 160 through the native mitral valve orifice 165 into the left ventricle 162. Once disposed in the left ventricle 162, the delivery device 150 may be at least partially unsheathed by translating a slideable sheath 152 in a proximal direction relative to the delivery device 150, as shown in FIG. 2B. The slideable sheath 152 may uncover the ventricular portion 103 of the expandable outer frame 108 of the prosthetic heart valve 100, which may cause the ventricular portion 103 to expand while the atrial portion 105 remains at least partially disposed within the delivery device 150 in a collapsed state. With the ventricular portion 103 in an expanded state, the delivery device 150 may be translated proximally such that the expanded portion of the prosthetic heart valve 100 contacts the ventricular side of the native valve orifice 165, as shown in FIG. 2B. The tines 109 on the ventricular portion 103 may contact the surrounding tissue of the native valve orifice 165 to anchor the ventricular portion 103 on the ventricular side of the native valve orifice 165, thus substantially anchoring the prosthetic heart valve 100 in place. The slideable sheath 152 may then uncover the remaining portion of the expandable prosthetic heart valve 100 (e.g., the atrial portion 105), allowing the atrial portion 105 to expand on the atrial side of the native valve orifice 165 such that the native valve orifice 165 is sandwiched by the ventricular and atrial portions 103, 105 of the prosthetic heart valve 100, as shown in FIG. 2C. The delivery device 150 may be retracted proximally when the prosthetic valve 100 is deployed and anchored in the native valve orifice 165, as shown in FIG. 2D.

In some embodiments, the ventricular and atrial anchors 103, 105 may be substantially disk-shaped when expanded and may have a relatively large diameter, for example about 50 mm or greater. When the prosthetic valve 100 is collapsed into the delivery device 150, the valve may have a diameter of about 11 mm or smaller. Collapsing the prosthetic valve 100 from a large size to a small size may require a relatively large amount of force. When loading the prosthetic valve 100 into the delivery device 150 outside the patient (e.g. in preparation for the heart valve replacement procedure), various mechanisms may be used to assist in the collapsing and/or to reduce the required forces. For example, the prosthetic valve 100 may be pulled through a funnel to assist with the collapse, and the loading may be performed in water or a solution having a low temperature (e.g. below the Af (austenite transformation finish) temperature of nitinol if the inner frame 106 and outer frame 108 are formed of nitinol). Even using these mechanisms, up to 50 lbs to 100 lbs or more of force may be required to collapse/load/sheathe the prosthetic heart valve 100 into the delivery device 150. If it is desired to try to re-collapse the prosthetic heart valve 100 after it has been partially deployed from the delivery device 150, for example in the condition shown in FIG. 2B (or just prior to the tines 109 engaging the native tissue), it may require significant force. Such re-collapsing may be desirable if the user wants to re-position the prosthetic heart valve 100, or even to remove the prosthetic heart valve 100 from the patient to abandon the procedure. It should be understood that the catheter/sheath of the delivery device 150 may be long and flexible, particularly if the delivery approach is transseptal, as the catheter/sheath needs to traverse the tortuous vasculature to reach the patient's heart. Any attempt to merely pull the partially-deployed prosthetic heart valve 100 back into the distal end of the sheath 152 may be extremely difficult, since large forces are required but the sheath which needs to handle those forces is long and flexible. However, embodiments described below may provide mechanisms and features that assist with re-collapse of the prosthetic heart valve 100 after it has been partially deployed, for example after the ventricular anchor 103 has been deployed.

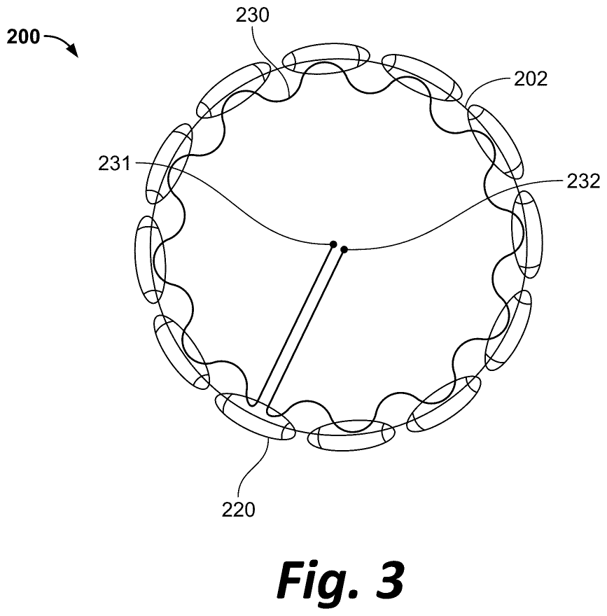

FIG. 3 illustrates a ventricular side of an embodiment of the prosthetic heart valve 200 including a mechanism in accordance with the disclosure for re-collapsing a prosthetic valve 200. The prosthetic valve 200 may be collapsed after it has been at least partly deployed in the patient but before final release of the valve 200 from the delivery apparatus to reposition the prosthetic valve 200 within the patient or remove the prosthetic valve 200 from the patient. The prosthetic heart valve 200 includes suture receiving rings 220 formed around the circumference of the ventricular end 202 of the valve 200. In the embodiment illustrated in FIG. 3, the prosthetic valve 200 includes twelve suture receiving rings 220 spaced equal distances apart around the circumference. It should be understood that prosthetic heart valve 200 may include twelve corresponding v-shaped elements. It is contemplated that the valve 200 may have a number of suture receiving rings greater or fewer than twelve disposed around the circumference of the ventricular end 202, and the rings may be spaced any distances apart (e.g., the prosthetic valve 100 illustrated in FIG. 1B has twenty four suture receiving rings 120 and twenty four corresponding v-shaped elements 111). Suture receiving rings 220 may be formed in the frame of the valve 200 at the apex of each ventricular v-shaped element (shown as 111 in FIG. 1B) on the ventricular end 202, similar to the tabs 112 formed on the atrial end 104 of prosthetic heart valve 100. The prosthetic heart valve 200 may further include a suture or other strand 230 threaded around the circumference of the ventricular end 202 of the valve 200 through each suture receiving ring 220, as is described below in greater detail. Strands 230 may be made of any suitable, sufficiently strong, sufficiently flexible, and sufficiently fine material and construction. Examples are wire (e.g., of nitinol), suture line, nylon line, or the like.

It should be understood that the prosthetic valve 200 may be a prosthetic mitral valve and will be described herein in relation to a delivery device using a transseptal method of delivery. The prosthetic valve 200 is not limited to being a mitral valve, nor is the prosthetic valve 200 limited to a transseptal method of delivery. The term “proximal” as used herein is used to describe a direction nearer the user of the delivery device when the device is used as intended in a transseptal procedure. The term “distal” as used herein is used to describe a direction farther away from the user of the delivery device when the device is used as intended in a transseptal procedure. While the prosthetic valve 200 is being deployed from the delivery device and implanted into the native valve orifice using a transseptal method of delivery, the atrial end 204 is proximal to the ventricular end 202 in relation to the delivery device. Stated another way, in a transseptal procedure, the ventricular end 202 is the leading end of the prosthetic valve 200 while the atrial end 204 is the trailing end.

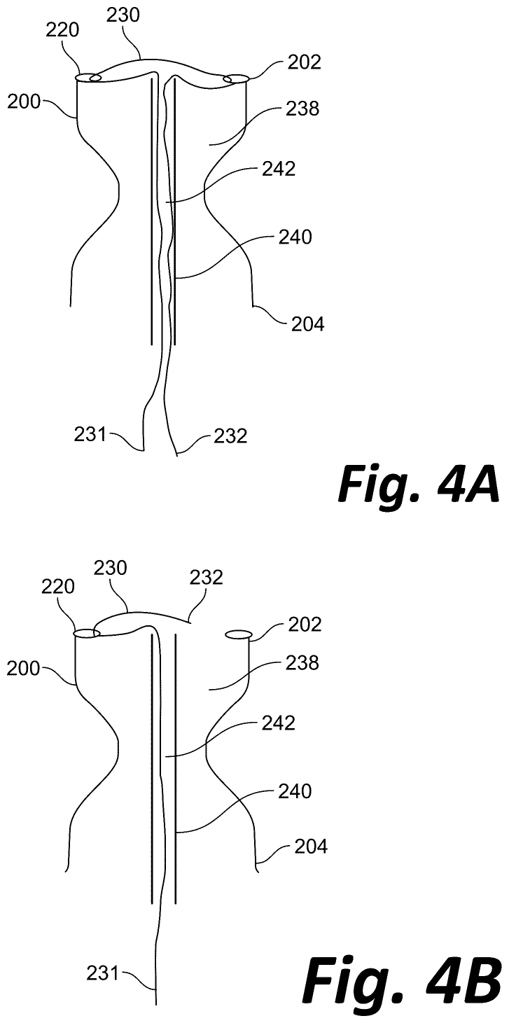

FIGS. 4A-B illustrate a schematic view of the prosthetic valve 200. As shown, the prosthetic valve 200 may be used in conjunction with a compression resistant member 240 extending through a central lumen 238 of the valve 200 in a longitudinal direction (i.e., from the atrial end 204 to the ventricular end 202). The compression resistant member 240 may be inserted into the central lumen 238 of the prosthetic valve 200 after the valve has been collapsed and loaded into a delivery device and the spacer described above has been removed from the prosthetic valve 200. In other examples, the compression resistant member 240 may be inserted into the central lumen 238 of the prosthetic valve 200 before the valve 200 is collapsed or as the valve 200 is collapsed and loaded into the delivery device. It should be noted that the compression resistant member 240 may extend longer than the length of the valve 200, such that the compression resistant member 240 may extend longitudinally beyond the ventricular end 202 of the prosthetic valve 200. The protrusion of the compression resistant member 240 beyond the ventricular end 202 of the prosthetic valve 200 may prevent the strand 230 from contacting and/or interfering with the prosthetic leaflets when the strand 230 is tensioned to collapse the prosthetic valve 200, as described below in greater detail. However, in other embodiments, the distal end of the compression resistant member 240 may axially align with, or be positioned proximal to, the ventricular end 202 of the prosthetic valve 200. The compression resistant member 240 may extend proximally from the ventricular end 202 of the prosthetic valve 200 to reach a handle on the proximal end of the delivery device for manipulation by a user. The strand 230 may be tensioned at the handle wherein the first and second ends 231, 232 are pulled simultaneously. Alternatively, the strand 230 may be tensioned by winding an end of the strand 230 (e.g., the second end 232) around the compression resistant member 240 and pulling on the opposing end (e.g., the first end 231). The compression resistant member 240 may be hollow, defining a lumen 242 therethrough. The compression resistant member 240 may be flexible and configured to resist buckling under a compressive force applied in the axial direction when the strand 230 is tensioned by the user, as described below in more detail. The compression resistant member 240 may be formed by any suitable material that will provide the above-described qualities, such as stacked coils, a cable tube, a hollow structural section (HSS) tube, a laser cut hypotube, a braided polymer shaft, or the like.

As shown in in FIGS. 4A-B, the strand 230 may extend through the lumen 242 of the compression resistant member 240 and through the suture receiving rings 220 on the ventricular end 202 of the prosthetic valve 200. In the illustrated embodiment, the strand 230 is a single continuous element. That is, the strand 230 extends from a first end 231 to a second end 232. Referring to FIG. 4A, the strand 230 may be threaded through the prosthetic valve 200 in a delivery configuration prior to the valve 200 being collapsed and loaded into the delivery device. In other examples, the strand 230 may be threaded through the prosthetic valve 200 after the valve 200 is collapsed and loaded into the delivery device. In the delivery configuration, the first end 231 of the strand 230 may be disposed outside of the patient's body and may be accessible to the user or a tool configured to be manipulated by the user (e.g. via attachment to a handle of the delivery device). From the first end 231, the strand 230 may extend distally through the delivery device and continue distally through the lumen 242 of the compression resistant member 240, e.g., from the atrial end 204 to the ventricular end 202. The strand 230 may emerge from the open distal end of the compression resistant member 240 on the ventricular end 202 of the valve 200 extending radially outward to a suture receiving ring 220, the strand 230 further extending approximately 360 degrees around the circumference of the ventricular end 202 passing through each consecutive suture receiving ring 220. After being threaded through each suture receiving ring 220, the strand 230 may extend radially inward to the compression resistant member 240 still on the ventricular end 202 and extend proximally from the ventricular end 202 to the atrial end 204 through the lumen 242 of the compression resistant member 240, continuing proximally through the delivery device to reach the second end 232. The second end 232 may be disposed proximate the first end 231 outside of the patient's body and accessible to the user or a tool configured to be manipulated by the user. It should be understood that, in FIG. 3, 231, 232 represent the points where the strand 230 enters the compression resistant member 240, with these points eventually leading to the first and second ends 231, 232, respectively. In FIGS. 4A-B, it should be understood that other components of the delivery system are omitted from the illustration, including for example an overlying sheath (e.g. retractable sheath 152) that may keep part or all of the prosthetic heart valve 200 in the collapsed delivery configuration prior to deployment.

The orientation of the strand 230 may be configured such that a user may apply a tension to the strand 230 by pulling both the first and second ends 231, 232 of the strand 230, resulting in a radially inward force applied to the suture receiving rings 220 through which the strand 230 is threaded, and thereby radially compressing the ventricular portion. Such tension may apply an axial force to the compression resistant member 240, and the structure of the compression resistant member 240 may be configured to prevent or substantially limit buckling or deformation of the compression resistant member 240, thus limiting axial compression of the prosthetic valve 100.

In the delivery configuration, the valve 200 may be configured to radially collapse on the ventricular end 202 of the valve 200. Such a configuration may allow the valve 200 to be at least partially deployed from the delivery device and re-collapsed for purposes of relocation or removal, as described below in greater detail with reference to the method of use. For example, after the ventricular end 202 has been deployed to an expanded configuration, but prior to the atrial end 204 being deployed to an expanded condition, the user may determine if the ventricular end is in a desired position. This may represent the condition illustrated in by prosthetic valve 100 in FIG. 2B, or a condition between those illustrated by prosthetic valve 100 in FIGS. 2A and 2B. If the position is not desirable, the user may pull the ends 231, 232 of the strand 230 proximally to force the ventricular end 202 to re-collapse, allowing the user to then re-position the ventricular end 202 prior to relaxing the strand 230 to allow the ventricular end 202 to re-expand. Or, if desired, the prosthetic heart valve 100, 200 can be re-sheathed while tension is maintained on the strand 230, and then the entire delivery device removed from the patient to abandon the procedure. After the valve 200 has been placed in a desired location, whether or not the strand 230 was used to re-collapse the ventricular end 202 to achieve the desired positioning, the strand 230 may be removed from the valve 200. Referring to FIG. 4B, the strand 230 may be removed by releasing the tension in one end of the strand 230 (e.g., the second end 232 as shown) and pulling the opposite end of the strand (e.g., the first end 231) such that the strand 230 follows the path of the first end 231 to decouple from the valve 200, as described below.

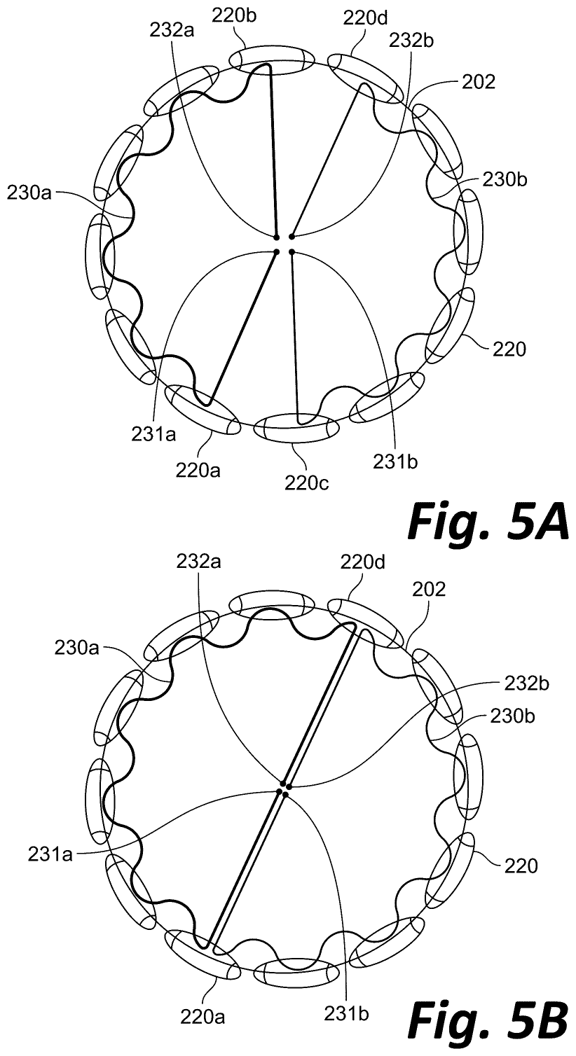

FIGS. 5A-B illustrate the ventricular side of the prosthetic heart valve 200 having an assembly for collapsing the ventricular end 202 of the valve 200 according to another embodiment of the disclosure. The embodiments shown in FIGS. 5A-B are substantially identical to the embodiment shown in FIG. 3, with the exception of the number and position of strands. As illustrated in FIGS. 5A-B, a first strand 230a and a second strand 230b may be used with the prosthetic valve 200. The first strand 230a may extend from a first end 231a to a second end 232a and the second strand 230b may extend from a first end 231b to a second end 232b. However, as with FIG. 3, the 231a-b and 232a-b labels in FIGS. 5A-B point to locations where the strands enter the compression resistant member 240, with those points eventually leading to the actual ends of the strands. In the delivery configuration, the ends 231a, 232a of the first strand 230a and the ends 231b, 232b of the second strand 230b may all be proximate to each other and extend outside the patient's body to be accessible to the user or a tool configured to be manipulated by the user (e.g. a handle of the delivery device). The first strand 230a may extend distally from the first end 231a through the delivery device, extending distally through the lumen 242 of the compression resistant member 240 (e.g., from the atrial end 204 to the ventricular end 202), the first strand 230a emerging from the lumen 242 and extending radially outwardly toward a suture receiving ring 220. The first strand 230a may further extend around approximately half of the circumference of the ventricular end 202 passing through each consecutive suture receiving ring 220, the first strand 230a continuing radially inwardly to the opening of the compression resistant member 240 on the ventricular side to extend proximally through the lumen 242 and reach the second end 232a of the first strand 230a.

The second strand 230b may have a substantially similar orientation to the first strand 230a, the second strand 230b extending distally from the first end 231b through the lumen 242, extending radially outwardly from the lumen 242 to a suture receiving ring 220 and extending around approximately half of the circumference of the ventricular end 202 through consecutive suture receiving rings 220 (e.g., the portion of the circumference and suture receiving rings 220 the first strand 230a does not pass through), the second strand 230b continuing radially inwardly to the opening of the compression resistant member 240 on the ventricular side to extend proximally through the lumen 242 to reach the second end 232b of the second strand 230b.

The strands 230a, 230b are not limited to which suture receiving rings 240 they extend to and pass through when emerging from and returning to the lumen 242 of the compression resistant member 240. For example, in the embodiment shown in FIG. 5A, the first strand 230a extends distally from the first end 231a, through the lumen 242 of the compression resistant member 240 and radially outward to a first suture receiving ring 220a. The first strand 230a then extends in a clockwise direction passing through each consecutive suture receiving ring 220 and returns to the compression resistant member 240 from a second suture receiving ring 220b. The second strand 230b extends distally from the first end 231b, through the lumen 242 of the compression resistant member 240 and radially outward to a third suture receiving ring 220c. The second strand 230b then extends in a counter-clockwise direction passing through each consecutive suture receiving ring 220 and returns to the compression resistant member 240 from a fourth suture receiving ring 220d. In other words, in the embodiment illustrated in FIG. 5A, each strand 230a, 230b extends through six consecutive suture receiving rings 220 (e.g. half the total number of suture receiving rings) without any suture receiving ring 220 receiving both of the strands 230a, 230b therethrough.

In other examples, such as the embodiment shown in FIG. 5B, the first and second strands 230a, 230b overlap and extend to and from the compression resistant member 240 via the same suture receiving rings 220a, 220d. That is, the first strand 230a extends distally from the first end 231a, through the lumen 242 of the compression resistant member 240, and extends radially outwardly to the first suture receiving ring 220a. The first strand 230a then extends clockwise around the circumference of the ventricular end 202 passing through each consecutive suture receiving ring 220, and continuing radially inward to the compression resistant member 240 from the fourth suture receiving ring 220d. The second strand 230b extends distally from the first end 231b, through the lumen 242 of the compression resistant member 240, and extends radially outwardly to the first suture receiving ring 220a. The second strand 230b then extends counter-clockwise around the circumference of the ventricular end 202 passing through each consecutive suture receiving ring 220, and continuing radially inward to the compression resistant member 240 from the fourth suture receiving ring 220d. In other words, in the embodiment illustrated in FIG. 5B, each strand 230a, 230b extends through seven consecutive suture receiving rings 220 (e.g. one more than half the total number of suture receiving rings) with two of the suture receiving rings 220a, 220d receiving both strands 230a, 230b therethrough, and the remaining suture receiving rings only receiving one strand therethrough.

It is contemplated that any number of strands may be used to collapse the ventricular end of a prosthetic valve. For example, a prosthetic valve may include one strand to be threaded through each suture receiving ring disposed on the ventricular end of the prosthetic valve. In other words, in the embodiment shown in FIG. 1B wherein the outer frame 108 of the prosthetic valve 100 includes 24 suture receiving rings 120, the valve 100 may include 24 strands wherein one strand is threaded through each suture receiving ring 120 and has first and second ends accessible to a user or operator. However, any number of strands between one strand and a number of strands that matches the total number of suture receiving rings 120 may be suitable. The use of more than one strand may reduce the amount of tension carried by any single strand when the strands are forcing the ventricular end to re-collapse, but the tradeoff may be additional volume of material and additional complexity of additional suture strands.

A method of collapsing a valve that has been at least partially deployed from a delivery device is described herein. When the prosthetic valve 200 is prepared in the delivery configuration as described above, the prosthetic valve 200 may be compressed and loaded into the delivery device in a collapsed condition. The delivery device may then be navigated through a patient to transport the collapsed valve 200 to the deployment site, e.g., the native mitral valve orifice. The prosthetic heart valve 200 may then be at least partially deployed from the delivery device to be positioned in the native valve orifice. At least a portion of the prosthetic heart valve 200 may radially expand upon deployment from the delivery device. For example, the ventricular portion of the prosthetic heart valve 200 may be deployed first and may radially expand while the atrial portion remains in the delivery device in a collapsed condition, as described above and shown in FIG. 2B. After the prosthetic valve 200 has been partly deployed in the patient and the location of the valve does not appear (e.g., fluoroscopically) to be as desired, the valve may be re-collapsed by tensioning the strand 230 by simultaneously pulling on the first and second ends 231, 232 of the strand 230. As described above, the tension in the strand 230 may cause a force on the suture receiving rings 220 directed radially inwardly, thereby radially collapsing the ventricular portion of the prosthetic heart valve 200. If multiple strands are utilized, both ends of each strand may be tensioned to cause the radial collapse. The prosthetic valve 200 may then be either removed from the patient (by withdrawing the delivery apparatus from the patient), or the valve 200 may be relocated in the patient (by manipulating the delivery apparatus to produce such relocation). It is contemplated that the ventricular portion of the prosthetic heart valve 200 may be collapsed by tensioning the strand 230, expanded by releasing the tension in the strand 230, and subsequently collapsed again so long as the strand 230 remains attached to the suture receiving rings 220. That is, it should be understood that even after barbs or tines (e.g., 109) have anchored into the surrounding tissue of the native valve, it may be possible to re-collapse the ventricular portion of the prosthetic heart valve 200 so long as the strand 230 remains in the delivery configuration attached to the suture receiving rings 220.

Assuming that valve relocation is the objective, when the prosthetic valve 200 is at the new location, it can be expanded again by releasing the tension on the strand 230. When the prosthetic valve 200 is satisfactorily positioned in the patient, the strand 230 may be released and decoupled from the prosthetic valve 200 by pulling either the first end 231 of the strand 230 or the second end 232 of the strand (but not both) proximally until the opposite end of the strand travels through the suture receiving rings 220 and the lumen 242 of the compression resistant member 240 to release contact with the prosthetic valve 200. For embodiments of the valve 200 including two strands 230a, 230b (or more), the same principle as described above will apply, wherein the operator may re-collapse the ventricular portion of the valve by pulling the first and second ends 231a, 232a, 231b, 232b of at least one of the first and second strands 230a, 230b. The first strand 230a may be released by pulling on either the first end 231a or the second end 232a (but not both). Similarly, the second strand 230b may be released by pulling on either the first end 231b or the second end 232b (but not both). The compression resistant member 240 may be removed from the prosthetic valve 200 with the delivery device after the prosthetic valve 200 is deployed and the delivery device is retracted.

According to one aspect of the disclosure, a prosthetic heart valve system comprises:
a collapsible and expandable outer frame extending from an atrial end to a ventricular end, the atrial and ventricular ends coupled by a central portion, the outer frame including a plurality of suture receiving rings formed around a circumference of the ventricular end;
a collapsible and expandable inner frame positioned radially inward of the outer frame and coupled to the outer frame;
a prosthetic valve assembly coupled to, and positioned radially inward of, the inner frame; and
a strand extending from a first free end to a second free end, a middle portion of the strand passing through each of the suture receiving rings in a delivery configuration of the prosthetic heart valve system, the first and second free ends configured to be pulled simultaneously by a user to radially collapse the ventricular end of the outer frame; and/or
a compression resistant member positioned radially inward of the prosthetic valve assembly in the delivery configuration of the prosthetic heart valve system; and/or
the compression resistant member defines a central lumen and is configured to resist an axial force applied to the compression resistant member when the first and second free ends are pulled by the user; and/or
in the delivery configuration of the prosthetic heart valve system, the middle portion of the strand extends through the central lumen of the compression resistant member, a distal open end of the compression resistant member extending distal to the outer frame; and/or
the strand is configured to be removed from the suture receiving rings when the strand is pulled by only one of the first and second free ends; and/or
the strand extends about 360 degrees around the circumference of the ventricular end of the outer frame; and/or
the outer frame includes twelve suture receiving rings disposed around the circumference of the ventricular end; and/or
in the delivery configuration, the strand passes through at least one of the suture receiving rings two times.

According to another aspect of the disclosure, a prosthetic heart valve system comprises:
a collapsible and expandable outer frame extending from an atrial end to a ventricular end, the atrial and ventricular ends coupled by a central portion, the outer frame including a plurality of suture receiving rings formed around a circumference of the ventricular end;
a collapsible and expandable inner frame positioned radially inward of the outer frame and coupled to the outer frame;
a prosthetic valve assembly coupled to, and positioned radially inward of, the inner frame;
a first strand extending from a first free end to a second free end, a middle portion of the first strand passing through a first group of the suture receiving rings in a delivery configuration of the prosthetic heart valve system; and
a second strand extending from a first free end to a second free end, a middle portion of the second strand passing through a second group of the suture receiving rings in the delivery configuration of the prosthetic heart valve system, the first and second ends of the first and second strands being configured to be simultaneously pulled by a user to radially collapse the ventricular end of the outer frame; and/or
every one of the plurality of suture receiving rings is part of the first group or the second group; and/or
the first group of suture receiving rings and the second group of suture receiving rings have no suture receiving rings in common; and/or
a first suture receiving ring at a first terminal end of the first group is adjacent a first suture receiving ring at a first terminal end of the second group; and/or
a second suture receiving ring at a second terminal end of the first group is adjacent a second suture receiving ring at a second terminal end of the second group; and/or
the first group and the second group have a first ring in common located at a first terminal end of the first and second groups, respectively; and/or
the first group and the second group have a second ring in common located at a second terminal end of the first and second groups, respectively; and/or
the first and second groups of suture receiving rings have two rings in common; and/or
the two rings in common between the first and second groups define terminal ends of each group of suture receiving rings; and/or
a compression resistant member positioned radially inward of the prosthetic valve assembly in the delivery configuration of the prosthetic heart valve system; and/or
the compression resistant member defines a central lumen and is configured to resist an axial force applied to the compression resistant member when the first and second ends of the first and second strands are pulled by a user; and/or
in the delivery configuration of the prosthetic heart valve system, the middle portion of the first strand and the middle portion of the second strand extend through the central lumen of the compression resistant member, a distal open end of the compression resistant member extending distal to the outer frame; and/or
the first strand passes through a first half of the plurality of suture receiving rings disposed on the ventricular end and the second strand passes through a second half of the plurality of suture receiving rings disposed on the ventricular end.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of deploying a prosthetic heart valve, comprising:
navigating a delivery device through a vasculature of a patient and to a native valve annulus while an outer frame of the prosthetic heart valve and an inner frame of the prosthetic heart valve are in a collapsed configuration within a slidable sheath of the delivery device;
partially deploying a ventricular end of the outer frame from the delivery device so that the ventricular end radially expands;
tensioning a strand that i) emerges radially outward from an open distal end of a compression resistant member, ii) is coupled to the prosthetic heart valve and extends around a circumference of the ventricular end of the outer frame, and iii) extends radially inward and through the open distal end of the compression resistant member;
radially collapsing the ventricular end of the outer frame by applying a radially inward force on the ventricular end of the outer frame by tensioning the strand;
after radially collapsing the ventricular end of the outer frame, repositioning the prosthetic heart valve relative to the native valve annulus;
releasing the tension on the strand to deploy the ventricular end so that the ventricular end radially expands; and
deploying the atrial end of the outer frame from the delivery device so that the atrial end of the outer frame radially expands.

2. The method of claim 1, wherein, while the strand is tensioned, the compression resistant member resists an axial compressive force exerted on the compression resistant member by the strand.

3. The method of claim 1, wherein the axial compressive force is exerted on the compression resistant member by both:
a first portion of the strand that emerges radially outward from the open distal end of the compression resistant member; and
a second portion of the strand that extends radially inward and through the open distal end of the compression resistant member.

4. The method of claim 3, wherein the strand is:
threaded distally through a lumen of the compression member; and
threaded proximally through the lumen of the compression resistant member.

5. The method of claim 1, wherein the ventricular end of the outer frame comprises a plurality of suture receiving rings.

6. The method of claim 5, wherein the strand is threaded:
through a first suture receiving ring of the plurality of suture receiving rings;
consecutively through each intermediate suture receiving ring of the plurality of suture receiving rings and around the circumference of the ventricular end of the outer frame; and
through a last suture receiving ring of the plurality of suture receiving rings.

7. The method of claim 6, wherein the last suture receiving ring and the first suture receiving ring are the same suture receiving ring.

8. The method of claim 1, wherein the strand is a single continuous element having a first end and a second end.

9. The method of claim 8, wherein tensioning the strand comprises pulling both the first end and the second end of the strand.

10. The method of claim 8, wherein the first end of the strand is wound around the compression resistant member.

11. The method of claim 1, wherein the strand extends about 360 degrees around the circumference of the ventricular end of the outer frame.

12. The method of claim 1, wherein the compression resistant member is positioned distally beyond the ventricular end of the outer frame while the strand is tensioned.

13. The method of claim 1, wherein the compression resistant member comprises one of: stacked coils, a cable tube, a hollow structural section (HSS) tube, a laser cut hypotube, or a braided polymer.

14. The method of claim 1, wherein navigating the delivery device through the vasculature of the patient to the native valve annulus occurs while the compression resistant member is positioned in a central lumen of the prosthetic heart valve.

15. The method of claim 14, wherein the compression resistant member is positioned in the central lumen of the prosthetic heart valve after the prosthetic heart valve is loaded into the delivery device.

16. The method of claim 1, wherein navigating the delivery device through the vasculature of the patient to the native valve annulus occurs while the inner frame is positioned radially within and coupled to the outer frame in the collapsed configuration.

17. The method of claim 16, wherein one or more prosthetic leaflets are coupled to the inner frame in the collapsed configuration.

18. The method of claim 1, further comprising:
before deploying the atrial end of the outer frame from the delivery device, translating the delivery device proximally so that the deployed ventricular end contacts a ventricular side of the native valve annulus.

19. The method of claim 1, wherein partially deploying the ventricular end of the outer frame from the delivery device occurs while the atrial end of the outer frame remains disposed within the delivery device in the collapsed configuration.

20. The method of claim 1, wherein deploying the ventricular end and the atrial end includes translating the slidable sheath.
